(19) United States
(12) Patent Application Publication
(10) Pub. No.: US 2010/0262053 A1
(43) Pub. Date: Oct. 14, 2010

(54) Apparatus and method for a universal patient controlled medical binder

(76) Inventor: Michael Gerard Crago, Littleton, CO (US)

Correspondence Address: Michael G. Crago, P.O Box 270102, Littleton, CO, 80127, US

(21) Appl. No.: 12/386,141

(22) Filed: Apr. 14, 2009

(51) Int. Cl. A61F 5/02

(52) U.S. Cl. 602/6

(57) ABSTRACT

A universal medical binder that is patient controlled, restoring maximum stability to a specific body part. The device is constructed of a unique material, making it possible for a hook type lock material (Velcro) to be attached on either side. The binder has a series of loop rings on the back that allows the material to be directed through the loop rings and then be folded back over itself to minimize the size of the device to accommodate a wide range of body parts. The specially designed loop rings act like a block and tackle system reducing the amount of strength required by the patient to activate the device and maximize support to a specific body part.

### RELATED APPLICATIONS

The present application is related to U.S. Pat. No. 4,641,642, issued Feb. 10, 1987, for PATIENT ACTIVATED BODY IMMOBILIZER, by williams, included by reference herein.

The present application is related to U.S. Pat. No. 4,273,130, issued Dec. 11, 1981, for CONSTRICTOR, by Simpson, included by reference herein.

The present application is related to U.S. Pat. No. 4,390,014, issued Jun. 28, 1983, for SELF RETAINING RIB BELT, by Forman, included by reference herein.

The present application is related to U.S. Pat. No. 2,900,976, issued Aug. 25, 1959, for MOVING HARNESS, by Kilmer, included by reference herein.

The present application is related to U.S. Pat. No. 5,363,523, issued Mar. 14, 1994, for SURGICAL PILLOW WITH REMOVABLE WEIGHT, by Blackburn, included by reference herein.

### FIELD OF THE INVENTION

The present invention relates to an adjustable universal medical binder and, more particularly, to a medical device that is patient controlled allowing the patient to activate as needed to restore anatomic stability to a specific body part and promote faster healing with fewer complications.

### BACKGROUND OF THE INVENTION

Since the inception of open thoracic surgery there has been a need for a device that could support the thoracic cavity, restore patient confidence, reduce pain, shorten rehabilitation time, and minimize post operative complications due to sternal instability.

Patients that have undergone medical procedures such as an open heart or a thoracic surgery can experience not only extreme pain, but also the fear that their chest is going to split open. this fear and anxiety can greatly affect the patient's ability to rehabilitate as these patient's are required to cough to clear fluid from their lungs as well as practice breathing exercises to ensure that their lung capacity is near pre-operative levels prior to discharge from the hospital. parients are required to practice incentive spirometry and range of motion excercises immediately after surgery in order to enhance peripheral circulation. the coughing and breathing exercises cause the chest cavity to expand, thus allowing the patient to feel the movement between the un-healed incision, creating fear and anxiety. open heart procedures inflict great trauma to an already diseased organ, in most cases requiring that the sternum be cut in half and spread open in order for the surgeon to access the heart muscle. the sternum is then wired back together and the outer layers of muscle and tissue are sutured accordingly. The rehabilitation and healing process may take up to 6-7 weeks depending on how compliant the patient is.

During the rehabilitation period there are risks of complications that can result in not only wound morbity but patient mortality. The expansion of the chest during coughing in a small percentage of patients can lead to sternal dehiscence. Additional post operative complications are pneumonia, thrombus formation and a variety of infections that could potentially be reduced by minimizing sternal instability in these patients.

Certain inventors have developed more and more elaborate and complex modalities for stabilizing the chest. Such modalities are available to hospitals through distributors or direct from the manufacturer for example, in U.S. Pat. Nos. 4,641,642, 4,273,130, 4,396,013, 2,900,976, and 5,363,523. though such modalities have achieved popularity and commercial success, there has been a continuing need for improvement.

There are a variety of medical harnesses and devices that encircle specific body parts for example Williams U.S. Pat. No. 4,641,642, which discloses a device for temporary immobilizing a selected region of a patients upper body. There are several sizes needed to accommodate the normal size range of patients. Smaller sizes can be difficult for frail patients to utilize as they have compromised strength after surgery. The single sided loop material is very flimsy and tends to ride up the back of the patient, changing the location of support. U.S. Pat. No. 4,273,130 Simpson, teaches of various types of medical constrictors that are used to apply compression to a body part to control blood circulation. The flexible material having projections that allow the material to be attached via Velcro. This device provides constant tension to a specific body part and is designed to compress a body part to maintain consistent blood flow. This device is not dynamic in the respect that there is no way to control the amount of tension applied to a specific body part to accommodate various activities. U.S. Pat. No. 4,396,013 Hasslinger, represents a harness for guiding an ambulating patient comprising of a strap that is secured around the patient. This device doesn't provide any controllable support to stabilize a body part, it is strictly utilized by a caregiver to help support an ambulating patient. U.S. Pat. No. 2,900,976 Kilmer, claims a device for elevating an impaired limb, comprising of a band encircling a leg which is raised and lowered by a pulley supported cable. This device is intended for a non-ambulating patient to elevate a limb and re-direct blood flow. U.S. Pat. No. 5,363,523 Blackburn, a pillow with removable weight. This device is utilized by a patient post-operatively, squeezing the pillow when coughing or deep breathing. This device is squeezed applying pressure against the wound causing the defect to actually spread apart promoting movement within the wound. This device does not provide circumferential support which is the key factor in reducing potential complications and promoting faster healing in post operative patients.

It is therefore an object of the invention to . . . provide a medical binder that restore's stability to a specific body part.

It is another object of the invention to . . . provide a harness that is universal in construction, and can be adjusted to fit a wide range of patients.

It is another object of the invention to . . . provide a harness that is dynamic in action and patient controlled.

It is another object of the invention to . . . provide a harness that can restore patient confidence and promote faster rehabilitation.

It is another object of the invention to . . . reduce post-operative complications.

### SUMMARY OF THE INVENTION

In accordance with the present invention, there is provided . . . a universal medical binder that is patient controlled, restoring maximum stability to a specific body part. The device is constructed of a unique material, making it possible for a hook type lock material (Velcro) to be attached on either side. The device has a series of loop rings on the back that allows the material to be directed through the loop rings and then be folded back over itself to adjust the size of the device to accommodate a wide range of body parts. The specially designed loop rings work as a block and tackle system reducing the amount of strength required by the patient to activate the device and maximize support to a specific body part.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a front perspective view of an embodiment of the medical device of the present invention showing the device on a human body;

FIG. 2 is a rear perspective view of an embodiment of the device of the present invention illustrating the position of the device on the mid-back section of the human body;

FIG. 3 is a front perspective view of an embodiment of the device of the present invention illustrating the device positioned mid-sternum and being activated by a patient;

FIG. 4 is a rear perspective view of a medical device shown in FIG. 1, adjusted to a full open position for accommodating larger body parts;

FIG. 5 is a rear perspective view of a medical device shown in FIG. 1, adjusted to the medium size accommodating average size body parts;

FIG. 6 is a rear perspective view of a medical device shown in FIG. 1, adjusted to it's smallest size to accommodate very small body parts;

FIG. 7 is a top detail view of an universal squeeze handle with various attachment slots to accommodate, shoulder and circumferential body straps;

FIG. 8 is a front sectional view of a right side universal squeeze handle as shown in FIG. 1, illustrating attachment of shoulder strap and circumferential body strap attached;

FIG. 9 is a front sectional view of a left side universal squeeze handle as shown in FIG. 1, illustrating attachment of shoulder strap, circumferential body strap, and adjustable universal squeeze handle stabilizer strap;

FIG. 10 is a front sectional view of a circumferential body strap end, illustrating the hook lock connector;

FIG. 11 is a front exploded view of a removable hook lock protector;

FIG. 12 is a top partial view of a circumferential body strap end, illustrating the double hook lock connector with the removable hook lock protector in a parallel position over attachment point; and

FIG. 13 is a rear partial view of a back of the device as shown in FIG. 2, illustrating a close up of the shoulder strap, loop ring, and loop ring attachment points.

For purposes of clarity and brevity, like elements and components will bear the same designations and numbering throughout the Figures.

### DESCRIPTION OF THE PREFERRED EMBODIMENT

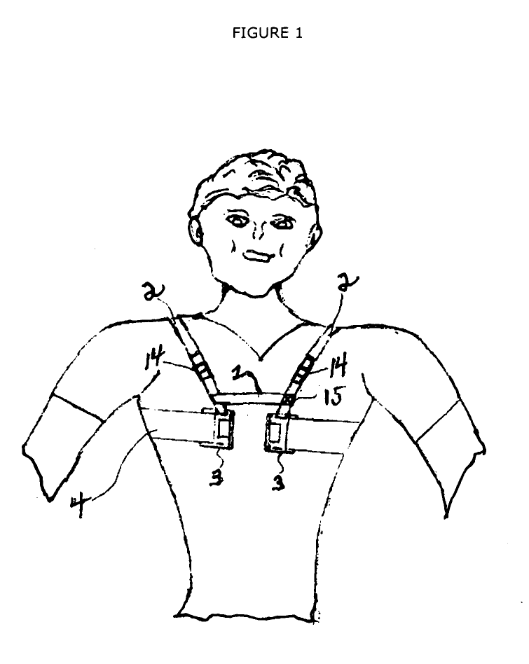

FIG. 1 is a front perspective view of an embodiment of the medical device of the present invention showing the device on a human body.

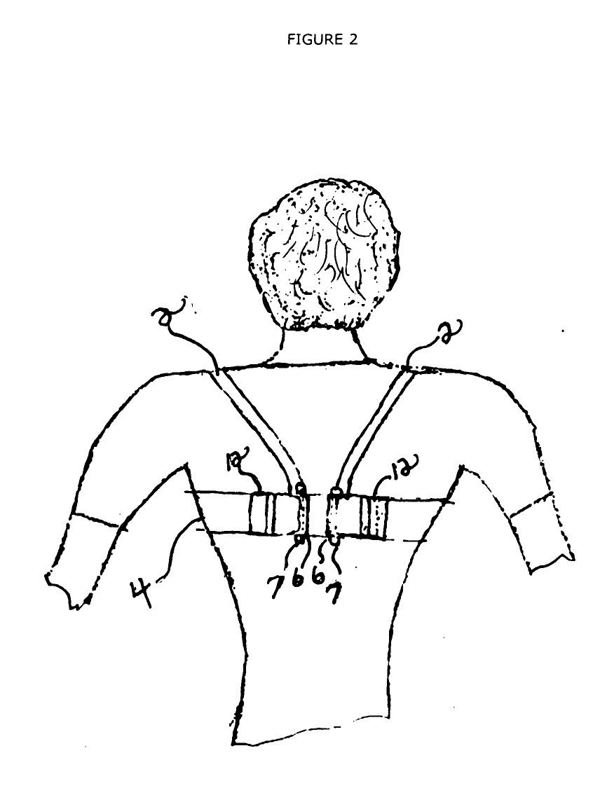

FIG. 2 is a rear perspective view of an embodiment of the device of the present invention illustrating the position of the device on the mid-back section of the human body.

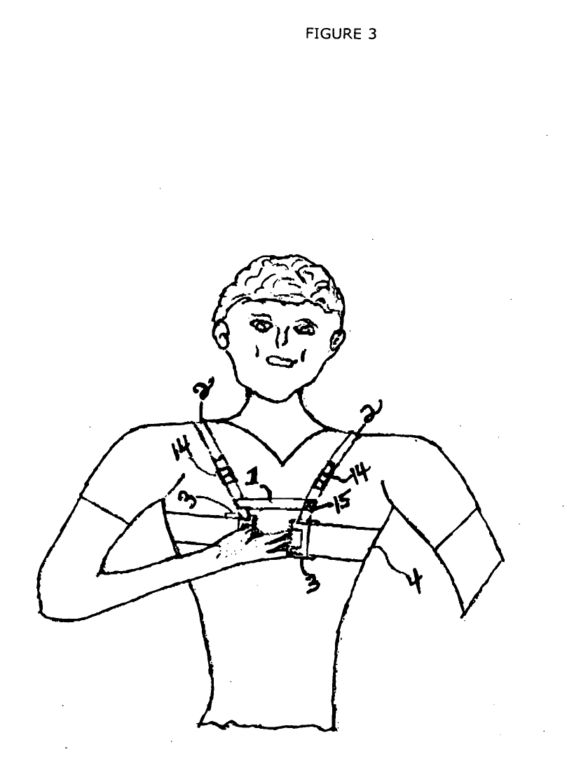

FIG. 3 is a front perspective view of an embodiment of the device of the present invention illustrating the device positioned mid-sternum and being activated by a patient.

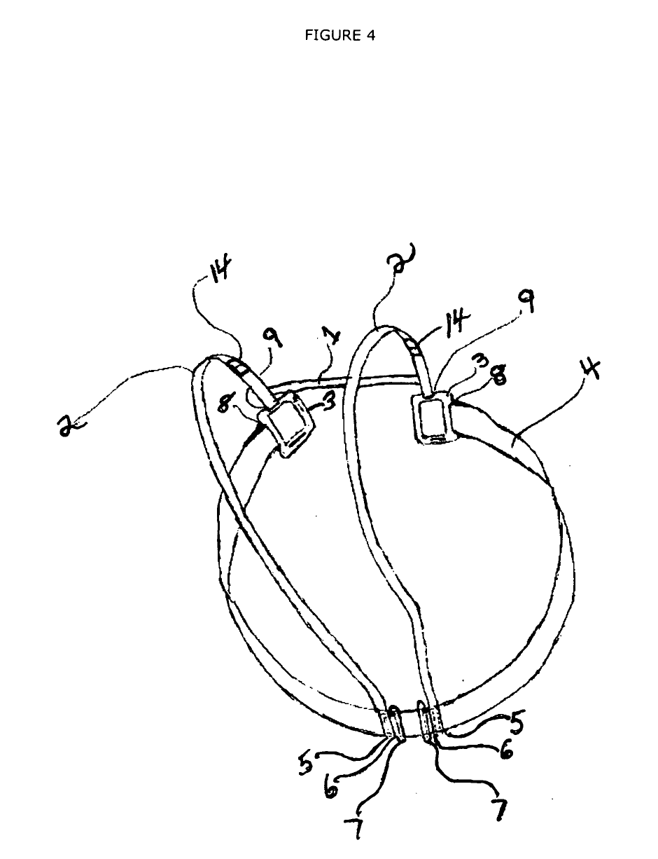

FIG. 4 is a rear perspective view of a medical device shown in FIG. 1, adjusted to a full open position accommodating larger body parts.

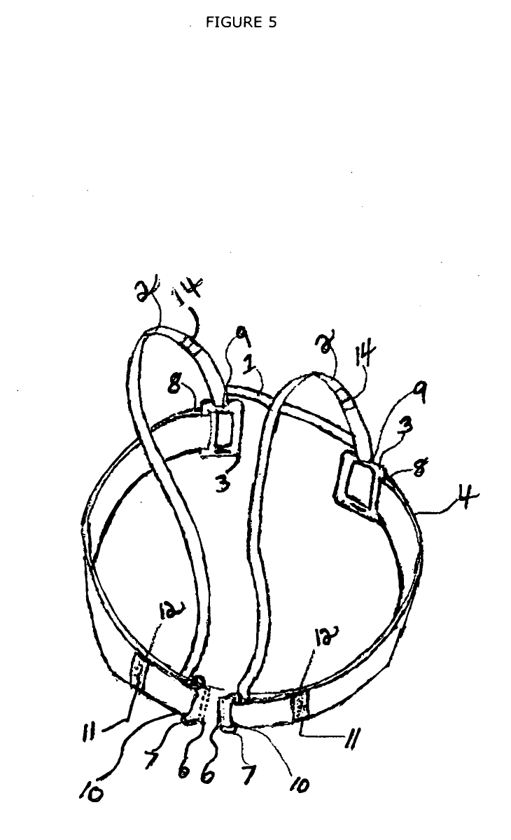

FIG. 5 is a rear perspective view of a medical device shown in FIG. 1, adjusted to the medium size accommodating average size body parts.

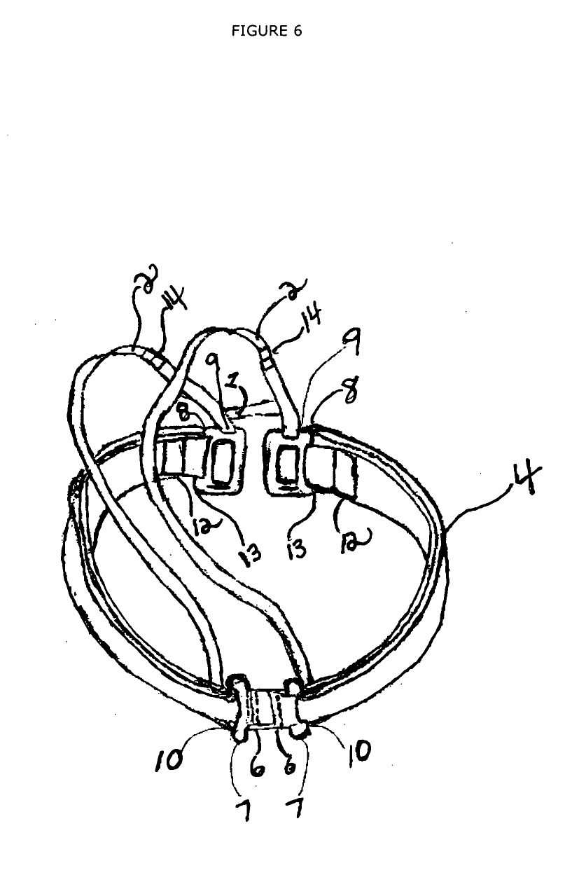

FIG. 6 is a rear perspective view of a medical device shown in FIG. 1, adjusted to it's smallest size to accommodate very small body parts.

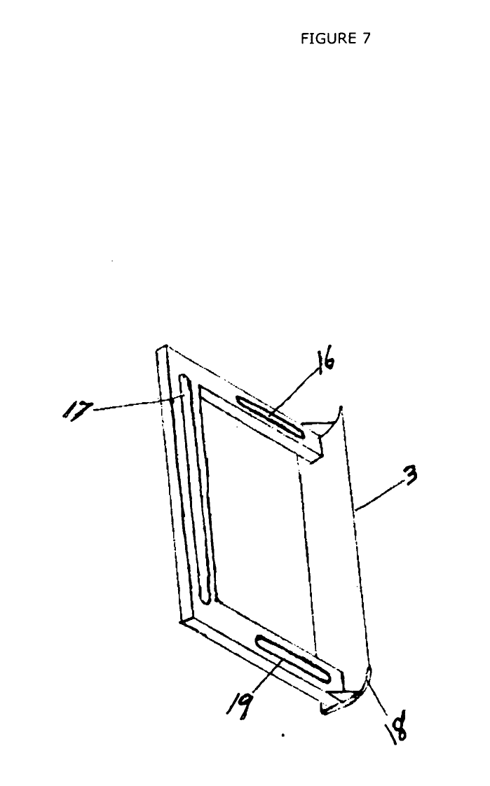

FIG. 7 is a top detail view of an universal squeeze handle with various attachment slots to accommodate, shoulder and circumferential body straps.

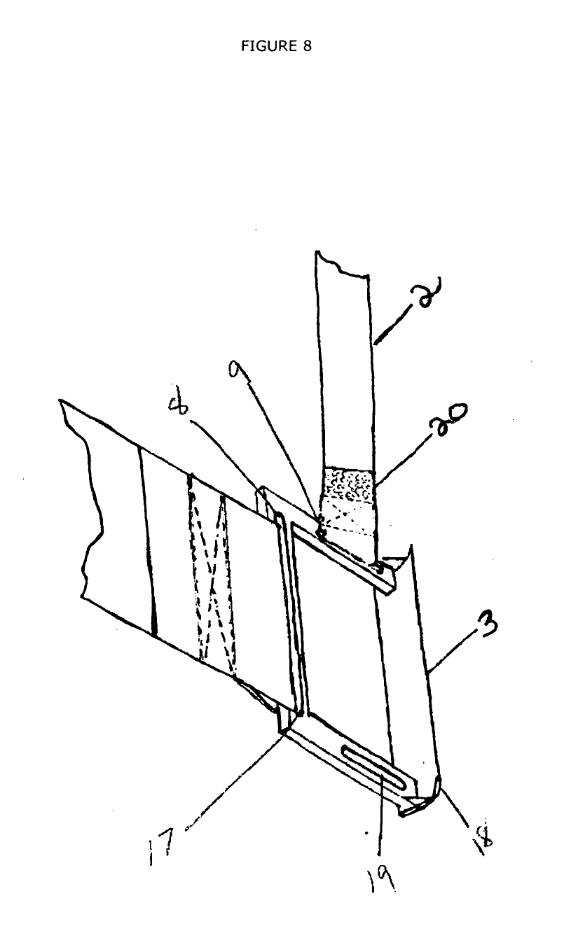

FIG. 8 is a front sectional view of a right side universal squeeze handle as shown in FIG. 1, illustrating attachment of shoulder strap 2 and circumferential body strap 4 attached.

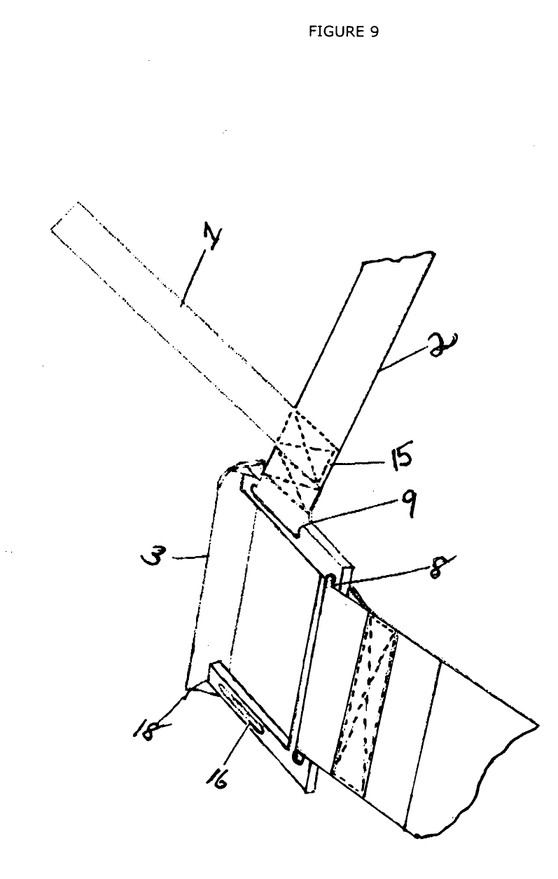

FIG. 9 is a front sectional view of a left side universal squeeze handle as shown in FIG. 1, illustrating attachment of shoulder strap 2, circumferential body strap 4, and adjustable universal squeeze handle stabilizer strap 1.

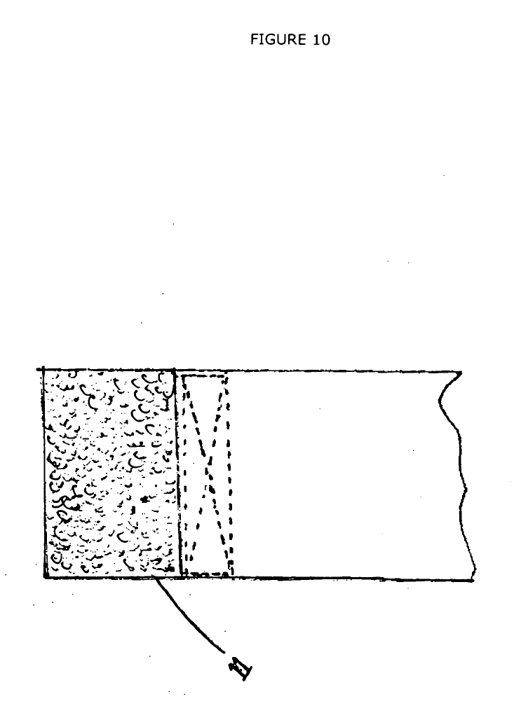

FIG. 10 is a front sectional view of a circumferential body strap 4 end, illustrating the hook lock connector.

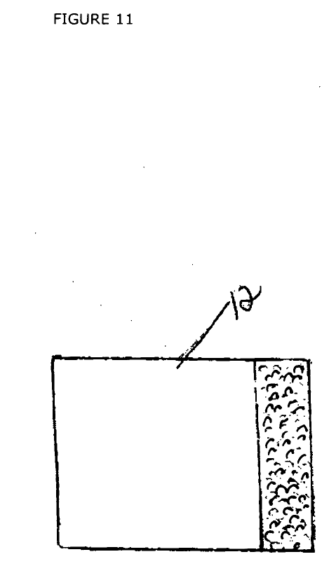

FIG. 11 is a front exploded view of a removable hook lock protector.

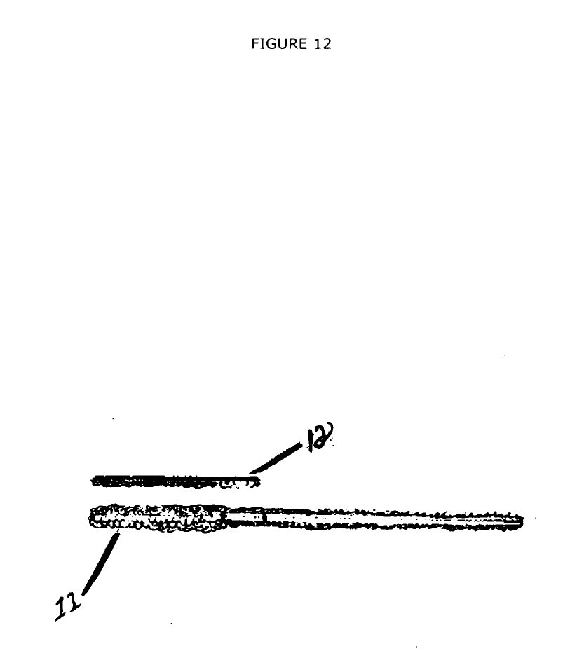

FIG. 12 is a top partial view of a circumferential body strap 4 end, illustrating the double hook lock connector with the removable hook lock protector in a parallel position over attachment point.

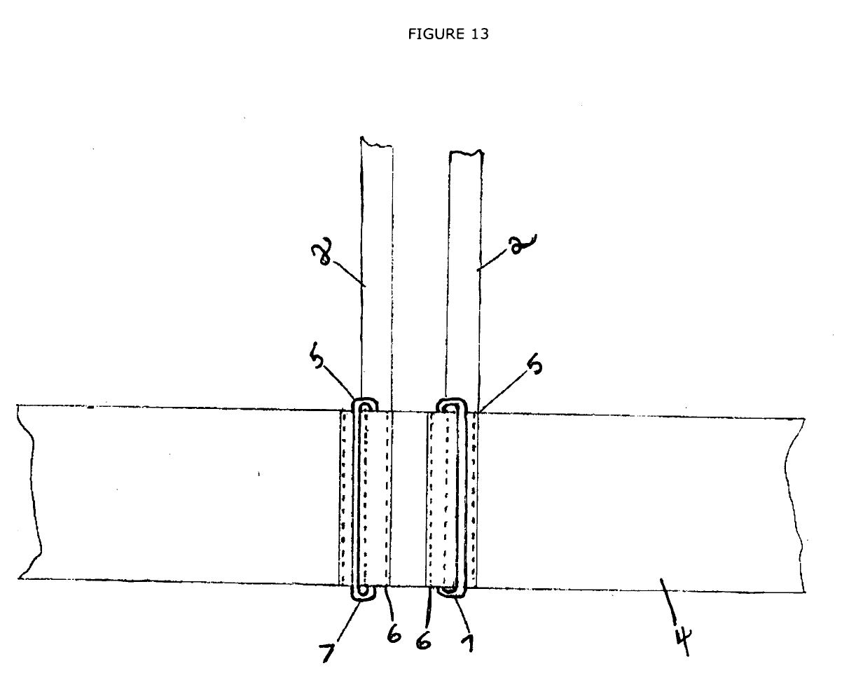

FIG. 13 is a rear partial view of an of the back of the device as shown in FIG. 2, illustrating a close up of the shoulder strap 2, loop ring, and loop ring attachment points.

Since other modifications and changes varied to fit particular operating requirements and environments will be apparent to those skilled in the art, the invention is not Considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. An apparatus and method for a universal patient controlled medical binder for supporting a specific body part, restoring anatomic stability, reducing pain, and promoting faster rehabilitation post surgery, comprising:
means for adjusting the squeeze handles to a desired width, allowing the patient to squeeze and activate at will;
means for securing the device over the shoulder and adjusting to the desired position over a specific body part;
means for allowing a patient to easily grab and squeeze, restoring stability to a specific body part, rigidly connected to said means for adjusting the squeeze handles to a desired width, allowing the patient to squeeze and activate at will;
means for providing a circumferential immobilizing strap that supports a specific body part;
means for easily disconnecting or connecting the adjustable shoulder straps, rigidly connected to said means for adjusting the squeeze handles to a desired width, allowing the patient to squeeze and activate at will;
means for attaching the loop rings to the rear of the device, rigidly connected to said means for providing a circumferential immobilizing strap that supports a specific body part;
means for providing an apparatus for allowing the circumferential body strap to feed through and double back on itself, rigidly connected to said means for attaching the loop rings to the rear of the device;
means for providing a protective cover over the exposed side of the double sided hook lock connector, located at the end of the circumferential body strap allowing the strap to pass through the loop rings and squeeze handles without catching up on the exposed loop material;
means for permanent connection of the shoulder strap to the device, rigidly connected to said means for providing a circumferential immobilizing strap that supports a specific body part;
means for attaching the circumferential body strap to the universal squeeze handles, removably connected to said means for providing a circumferential immobilizing strap that supports a specific body part, and removably connected to said means for allowing a patient to easily grab and squeeze, restoring stability to a specific body part;
means for attaching the front shoulder straps to the top of the universal squeeze handles;
means for allowing the circumferential body strap to be adjusted to a medium size;
means for enabling the double sided connector to attach to the circumferential body strap on either side;
means for allowing the circumferential body strap to double back through the universal squeeze handles and re-attach to itself, adjusting the device to the smallest size;
means for attachment of the right shoulder strap to a universal squeeze handle;
means for attaching the circumferential body strap to the universal squeeze handle;
means for creating an ergonomic space for a patients fingers to grip the universal squeeze handle;
means for attaching the left side adjustable shoulder strap to the top of the universal squeeze handle; and
means for attaching the adjustable squeeze handle stabilizer strap, adjusting to the desired width, lockably connected to said means for easily disconnecting or connecting the adjustable shoulder straps, and rigidly connected to said means for securing the device over the shoulder and adjusting to the desired position over a specific body part.

2. The apparatus and method for a universal patient controlled medical binder in accordance with claim 1, wherein said means for adjusting the squeeze handles to a desired width, allowing the patient to squeeze and activate at will comprises a self attaching adjustable universal squeeze handle stabilizer strap.

3. The apparatus and method for a universal patient controlled medical binder in accordance with claim 1, wherein said means for securing the device over the shoulder and adjusting to the desired position over a specific body part comprises a secures the device in place shoulder strap.

4. The apparatus and method for a universal patient controlled medical binder in accordance with claim 1, wherein said means for allowing a patient to easily grab and squeeze, restoring stability to a specific body part comprises an easy to grab, light weight, smooth universal squeeze handles.

5. The apparatus and method for a universal patient controlled medical binder in accordance with claim 1, wherein said means for providing a circumferential immobilizing strap that supports a specific body part comprises an adjusts to fit all sizes circumferential body strap.

6. The apparatus and method for a universal patient controlled medical binder in accordance with claim 1, wherein said means for easily disconnecting or connecting the adjustable shoulder straps comprises an allows the shoulder strap to be disconnected during application shoulder strap quick disconnects.

7. The apparatus and method for a universal patient controlled medical binder in accordance with claim 1, wherein said means for attaching the loop rings to the rear of the device comprises a location where loop rings attach loop ring attachment point.

8. The apparatus and method for a universal patient controlled medical binder in accordance with claim 1, wherein said means for providing an apparatus for allowing the circumferential body strap to feed through and double back on itself comprises an allow strap to loop back on itself loop rings.

9. The apparatus and method for a universal patient controlled medical binder in accordance with claim 1, wherein said means for providing a protective cover over the exposed side of the double sided hook lock connector, located at the end of the circumferential body strap allowing the strap to pass through the loop rings and squeeze handles without catching up on the exposed loop material comprises a protects the exposed side of hook lock connector removable hook lock covers.

10. The apparatus and method for a universal patient controlled medical binder in accordance with claim 1, wherein said means for permanent connection of the shoulder strap to the device comprises a rear shoulder strap attachment point.

11. The apparatus and method for a universal patient controlled medical binder in accordance with claim 1, wherein said means for attaching the circumferential body strap to the universal squeeze handles comprises a circumferential body strap entry point at universal squeeze handle.

12. The apparatus and method for a universal patient controlled medical binder in accordance with claim 1, wherein said means for attaching the front shoulder straps to the top of the universal squeeze handles comprises a front entry point for shoulder strap through universal squeeze handle.

13. The apparatus and method for a universal patient controlled medical binder in accordance with claim 1, wherein said means for allowing the circumferential body strap to be adjusted to a medium size comprises a loop ring insertion point for circumferential body strap.

14. The apparatus and method for a universal patient controlled medical binder in accordance with claim 1, wherein said means for enabling the double sided connector to attach to the circumferential body strap on either side comprises a connects on either side self adhering double back hook lock connector.

15. The apparatus and method for a universal patient controlled medical binder in accordance with claim 1, wherein said means for allowing the circumferential body strap to double back through the universal squeeze handles and re-attach to itself, adjusting the device to the smallest size comprises an insertion point for circumferential body strap looped back through universal squeeze handle.

16. The apparatus and method for a universal patient controlled medical binder in accordance with claim 1, wherein said means for attachment of the right shoulder strap to a universal squeeze handle comprises a right side adjustable shoulder strap slot.

17. The apparatus and method for a universal patient controlled medical binder in accordance with claim 1, wherein said means for attaching the circumferential body strap to the universal squeeze handle comprises a circumferential body strap slot.

18. The apparatus and method for a universal patient controlled medical binder in accordance with claim 1, wherein said means for creating an ergonomic space for a patients fingers to grip the universal squeeze handle comprises an universal squeeze handle finger grip.

19. The apparatus and method for a universal patient controlled medical binder in accordance with claim 1, wherein said means for attaching the left side adjustable shoulder strap to the top of the universal squeeze handle comprises a left side adjustable shoulder strap slot.

20. The apparatus and method for a universal patient controlled medical binder in accordance with claim 1, wherein said means for attaching the adjustable squeeze handle stabilizer strap, adjusting to the desired width comprises a hook lock connection for adjustable squeeze handle stabilizer strap.

21. An apparatus and method for a universal patient controlled medical binder for supporting a specific body part, restoring anatomic stability, reducing pain, and promoting faster rehabilitation post surgery, comprising:
a self attaching adjustable universal squeeze handle stabilizer strap, for adjusting the squeeze handles to a desired width, allowing the patient to squeeze and activate at will;
a secures the device in place shoulder strap, for securing the device over the shoulder and adjusting to the desired position over a specific body part;
an easy to grab, light weight, smooth universal squeeze handles, for allowing a patient to easily grab and squeeze, restoring stability to a specific body part, rigidly connected to said adjustable universal squeeze handle stabilizer strap;
an adjusts to fit all sizes circumferential body strap, for providing a circumferential immobilizing strap that supports a specific body part;
an allows the shoulder strap to be disconnected during application shoulder strap quick disconnects, for easily disconnecting or connecting the adjustable shoulder straps, rigidly connected to said adjustable universal squeeze handle stabilizer strap;
a location where loop rings attach loop ring attachment point, for attaching the loop rings to the rear of the device, rigidly connected to said circumferential body strap;
an allow strap to loop back on itself loop rings, for providing an apparatus for allowing the circumferential body strap to feed through and double back on itself, rigidly connected to said loop ring attachment point;
a protects the exposed side of hook lock connector removable hook lock covers, for providing a protective cover over the exposed side of the double sided hook lock connector, located at the end of the circumferential body strap allowing the strap to pass through the loop rings and squeeze handles without catching up on the exposed loop material;
a rear shoulder strap attachment point, for permanent connection of the shoulder strap to the device, rigidly connected to said circumferential body strap;
a circumferential body strap entry point at universal squeeze handle, for attaching the circumferential body strap to the universal squeeze handles, removably connected to said circumferential body strap, and removably connected to said universal squeeze handles;
a front entry point for shoulder strap through universal squeeze handle, for attaching the front shoulder straps to the top of the universal squeeze handles;
a loop ring insertion point for circumferential body strap, for allowing the circumferential body strap to be adjusted to a medium size;
a connects on either side self adhering double back hook lock connector, for enabling the double sided connector to attach to the circumferential body strap on either side;
an insertion point for circumferential body strap looped back through universal squeeze handle, for allowing the circumferential body strap to double back through the universal squeeze handles and re-attach to itself, adjusting the device to the smallest size;
a right side adjustable shoulder strap slot, for attachment of the right shoulder strap to a universal squeeze handle;
a circumferential body strap slot, for attaching the circumferential body strap to the universal squeeze handle;
an universal squeeze handle finger grip, for creating an ergonomic space for a patients fingers to grip the universal squeeze handle;
a left side adjustable shoulder strap slot, for attaching the left side adjustable shoulder strap to the top of the universal squeeze handle; and
a hook lock connection for adjustable squeeze handle stabilizer strap, for attaching the adjustable squeeze handle stabilizer strap, adjusting to the desired width, lockably connected to said shoulder strap quick disconnects, and rigidly connected to said shoulder strap.

22. The apparatus and method for a universal patient controlled medical binder as recited in claim 21, wherein said shoulder strap is easily disconnects during fitting.

23. The apparatus and method for a universal patient controlled medical binder as recited in claim 21, wherein said removable hook lock covers is removable.

24. The apparatus and method for a universal patient controlled medical binder as recited in claim 22, wherein said removable hook lock covers is removable.

25. An apparatus and method for a universal patient controlled medical binder for supporting a specific body part, restoring anatomic stability, reducing pain, and promoting faster rehabilitation post surgery, comprising:
a self attaching adjustable universal squeeze handle stabilizer strap, for adjusting the squeeze handles to a desired width, allowing the patient to squeeze and activate at will;
a secures the device in place, easily disconnects during fitting shoulder strap, for securing the device over the shoulder and adjusting to the desired position over a specific body part;
an easy to grab, light weight, smooth universal squeeze handles, for allowing a patient to easily grab and squeeze, restoring stability to a specific body part, rigidly connected to said adjustable universal squeeze handle stabilizer strap;
an adjusts to fit all sizes circumferential body strap, for providing a circumferential immobilizing strap that supports a specific body part;
an allows the shoulder strap to be disconnected during application shoulder strap quick disconnects, for easily disconnecting or connecting the adjustable shoulder straps, rigidly connected to said adjustable universal squeeze handle stabilizer strap;
a location where loop rings attach loop ring attachment point, for attaching the loop rings to the rear of the device, rigidly connected to said circumferential body strap;
an allow strap to loop back on itself loop rings, for providing an apparatus for allowing the circumferential body strap to feed through and double back on itself, rigidly connected to said loop ring attachment point;
a protects the exposed side of hook lock connector, removable removable hook lock covers, for providing a protective cover over the exposed side of the double sided hook lock connector, located at the end of the circumferential body strap allowing the strap to pass through the loop rings and squeeze handles without catching up on the exposed loop material;
a rear shoulder strap attachment point, for permanent connection of the shoulder strap to the device, rigidly connected to said circumferential body strap;
a circumferential body strap entry point at universal squeeze handle, for attaching the circumferential body strap to the universal squeeze handles, removably connected to said circumferential body strap, and removably connected to said universal squeeze handles;
a front entry point for shoulder strap through universal squeeze handle, for attaching the front shoulder straps to the top of the universal squeeze handles;
a loop ring insertion point for circumferential body strap, for allowing the circumferential body strap to be adjusted to a medium size;
a connects on either side self adhering double back hook lock connector, for enabling the double sided connector to attach to the circumferential body strap on either side;
an insertion point for circumferential body strap looped back through universal squeeze handle, for allowing the circumferential body strap to double back through the universal squeeze handles and re-attach to itself, adjusting the device to the smallest size;
a right side adjustable shoulder strap slot, for attachment of the right shoulder strap to a universal squeeze handle;
a circumferential body strap slot, for attaching the circumferential body strap to the universal squeeze handle;
an universal squeeze handle finger grip, for creating an ergonomic space for a patients fingers to grip the universal squeeze handle;
a left side adjustable shoulder strap slot, for attaching the left side adjustable shoulder strap to the top of the universal squeeze handle; and
a hook lock connection for adjustable squeeze handle stabilizer strap, for attaching the adjustable squeeze handle stabilizer strap, adjusting to the desired width, lockably connected to said shoulder strap quick disconnects, and rigidly connected to said shoulder strap.
